(19) United States
(12) Patent Application Publication
(10) Pub. No.: US 2006/0039977 A1
(43) Pub. Date: Feb. 23, 2006

(54) Oral drug delivery system

(76) Inventors: Mark Rupert Tucker, Chadlington (GB); Eamon Flahive, Wallingford (GB); Alistair Stokes, Maidenhead (GB); Jonathan Watts, Lambourne (GB)

Correspondence Address: STITES & HARBISON PLLC, 1199 NORTH FAIRFAX STREET, SUITE 900, ALEXANDRIA, VA, 22314, US

(21) Appl. No.: 10/470,711

(22) PCT Filed: Feb. 4, 2002
(86) PCT No.: PCT/GB02/00464

§ 371 Date: Sep. 8, 2005

(30) Foreign Application Priority Data
Feb. 2, 2001 (GB) 0102725.9

(51) Int. Cl. A61K 9/24

(52) U.S. Cl. 424/473

(57) ABSTRACT

There is disclosed an oral delivery system, which comprises: a sachet at least partially formed from at least one microporous permeable membrane, and defining a cavity; a physiologically active substance dissolved or dispersed in a liquid or gel, within the cavity, the microporous or permeable membrane being in contact with the liquid or gel and being permeable to the physiologically active substance in the liquid or gel; and an encapsulating layer surrounding the sachet; characterised in that either: a) the membrane is hydrophilic and the contents of the sachet are hydrophobic; or b) the membrane is hydrophobic and the contents of the sachet are hydrophilic; whereby, in use, the encapsulating layer is first dissolved in the gastro-intestinal tract (GIT) and thereafter passage of the physiologically active substance into the GIT through the membrane is rate-controlled. A method of manufacturing the oral delivery system is also disclosed.

This invention relates to a medical delivery system.

Various medical delivery systems are known, which include bandages or patches; and there have been numerous so-called nicotine patches marketed but many suffer from an inconsistent or incomplete transfer of the active ingredient (nicotine) to the person wearing the patch.

However, good results are obtainable by a patch-style system disclosed in United Kingdom Patent No. 2232892, which covers a body for the transdermal administration of a physiologically active substance, said body comprising an impermeable backing and a microporous or permeable membrane which define a cavity therebetween, said physiologically active substance being contained within said cavity in liquid form, said microporous or permeable membrane being permeable to and in contact with said physiologically active substance and the liquid material confined between said impermeable backing and said microporous or permeable membrane within said cavity being substantially immobilised by a viscous flowable gel, characterised in that either;
a) said membrane is hydrophilic and the contents of said cavity are hydrophobic; or
b) said membrane is hydrophobic and said cavity contains a hydrophilic wetting agent;
whereby, in use, passage of said physiologically active substance through said microporous or permeable membrane is rate-controlling and said physiologically active substance is released from said microporous or permeable membrane at a rate that is substantially constant over a period of hours.

According to the present invention, there is provided an oral delivery system, which comprises:
a sachet at least partially formed from at least one microporous or permeable membrane, and defining a cavity;
a physiologically active substance dissolved or dispersed in a liquid or gel, within the cavity, the microporous or permeable membrane being in contact with the liquid or gel and being permeable to the physiologically active substance in the liquid or gel; and
an encapsulating layer surrounding the sachet;
characterised in that either:
   a) the membrane is hydrophilic and the contents of the sachet are hydrophobic; or
   b) the membrane is hydrophobic and the contents of the sachet are hydrophilic;
whereby, in use, the encapsulating layer is first dissolved in the gastro-intestinal tract (GIT) and thereafter passage of the physiologically active substance into the GIT through the membrane is rate-controlled.

In practice, two membranes are brought together at their edge regions, with a cavity being left between the edge regions, into which cavity is introduced the physiologically active substance dissolved or dispersed in the liquid or gel. The resulting product is then sealed transversely at intervals and cut in the region of the seals, so as to form individual sachets. Each of the sachets is then encapsulated within a suitable encapsulating material, such as gelatin. The purpose of the encapsulating layer is to provide a dosing vehicle for the active formulation (i.e. the physiologically active substance within the sachet) to reach the intended site of action, for example the stomach, duodenum or bowel, before being digested too early.

The sachet is, as indicated above, essentially formed from two sheets of membrane which are joined at their edges and then joined at spaced transverse locations. The two sheets of membrane can be identical, and this is typical where the contents of the cavity are to include a single physiologically active agent. If, however, there are present within the cavity of the sachet two different physiologically active substances, it is possible for the two sheets of membrane from which the sachet is formed to be formed of two different materials which, bearing in mind the hydrophilic/hydrophobic relationship between the active substances and the membranes, can mean that the release characteristics of the different active substances vary considerably.

Examples of membrane materials which can be employed in the production of the sachet forming part of the oral delivery system of the present invention include polyethylene, polyvinyl acetate, copolymers of ethyl vinylacetate, polymethacrylate, polyvinyl chloride, ethylcelluloses, polyamides, polyurethanes, polyethers, and copolyesters, this list not being exhaustive. By using different membranes in the construction of the sachet forming part of the oral delivery system of the present invention, the system can deliver a mixture of drugs of widely different polarity, something which is difficult if not impossible to achieve with existing technologies. Each drug would migrate to that membrane to which it has the better affinity based on the hydrophilic/hydrophobic relationship, and the drug would then permeate through that membrane by the usual diffusion mechanism. The rate of diffusion could be tailored for each drug or other physiologically active substance by varying the chemistry of the membrane, its thickness, tortuosity and porosity, for example.

The encapsulating layer can be formed, as indicated above of gelatin or some other material familiar to those skilled in the art. If desired, the resulting capsule may optionally be enterically coated to provide a further sustained release function.

By means of the present invention it is possible to deliver a wide range of physiologically active substances, many of which are already delivered by known delivery systems. These include nitroglycerin and nicotine, amongst many.

For a better understanding of the present invention and to show how the same may be carried into effect, reference will now be made, by way of example, to the accompanying drawings, in which:

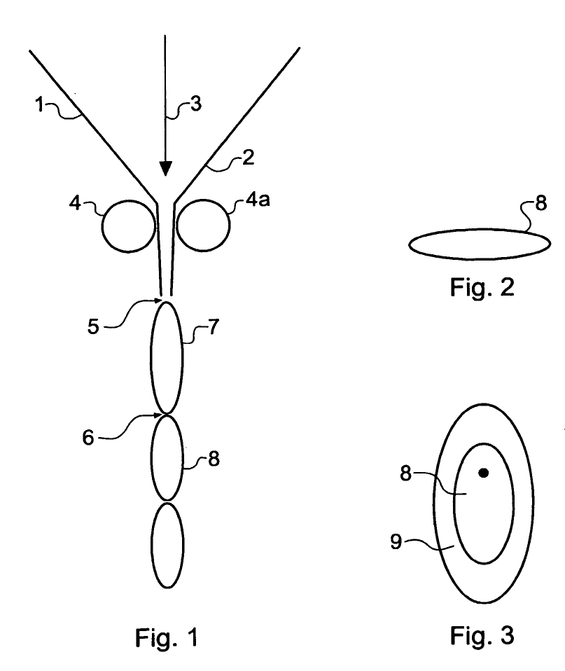

FIG. 1 shows schematically a production line for producing an oral delivery system in accordance with the present invention;

FIG. 2 shows a sachet as produced in accordance with the production line of FIG. 1; and

FIG. 3 shows an encapsulated sachet.

Referring to FIG. 1, two membranes 1, 2 are drawn from separate sources and are brought together, with the gel/drug combination being introduced into the space between the membranes from a source 3. The two membranes 1, 2 are passed between two heated sealing rollers 4, 4a which join the edge regions so as to form a type of container having a cavity containing the gel/drug combination. The product is then passed through a transverse heating station 5 which effectively forms a transverse seal which is to form the bottom sealed region of an upper container and the upper sealed region of a lower container, and the thus sealed sachets 7 are then moved to a cutting station 6 where they are cut into individual sachets 8. One such sachet is shown in FIG. 2. The sachet of FIG. 2 is then encapsulated within a gelatin layer 9 to give the product shown in FIG. 3.

The present invention will now be illustrated by the following example.

### EXAMPLE

The following two formulations were prepared

| Formulation A | Formulation B |
|---|---|
| Core: | Core: |
| Nicotine 10 mg | Nicotine 10 mg |
| Carboxy methyl cellulose 2 mg | Carboxy methyl cellulose 2 mg |
| Water 38 mg | Water 38 mg |
| Sachet: | Sachet: |
| CoTran 19% eva membrane | Bertek Medfilm 325 membrane |

Both formulations were tested for release of the drug in vitro and depending on the membrane material chosen, different release profiles were obtained. In this example, zero order release characteristics were demonstrated for Formulation A for up to 6 hours approximately. Formulation B did not offer zero order release. Nicotine is presented by way of example only and the invention is not limited to this drug. It is envisaged that many drugs from a host of therapeutic categories may be delivered by this technology.

The present invention envisages that the sachet containing the drug will be placed inside a capsule which will degrade in the GIT after a period of time has elapsed, thereby facilitating release of drug in a controlled manner. An example of the capsule material could consist of hard or soft gel; and the resulting gelatin capsules optionally may be film coated. The use of soft gel for the gelatin capsules is not limited to hydrophobic liquids (as is normally the case because water or water-soluble contents can interact with the shell).

Set out below, purely for example, are typical physical dimensions of materials used in the oral sachets.
Membrane material:
thickness: 0.5 to 3 mils (milli-inches)
composition: selected from a range of polymer materials as listed above
porosity: can range from non-porous to porous.
Drug content:
Determined by therapeutic dose requirements
   e.g. Nicotine: 10 to 30 mg
   Diclofenac: 10 to 100 mg
Surface area of sachet: 0.5 cm² to 4 cm²
Capsule material: hard shell gelatin, or soft gel gelatin.
Film coating material: any standard material used in the industry such as OPADRY® system. Additionally, more complex control release agents such as SURELEASE® may optionally be employed either inside or outside the sachet.

1. An oral delivery system, which comprises:
a sachet at least partially formed from at least one microporous or permeable membrane, and defining a cavity;
a physiologically active substance dissolved or dispersed in a liquid or gel, within the cavity, the microporous or permeable membrane being in contact with the liquid or gel and being permeable to the physiologically active substance in the liquid or gel; and
an encapsulating layer surrounding the sachet;
characterized in that either:
a) the membrane is hydrophilic and the contents or the sachet are hydrophobic; or
b) the membrane is hydrophobic and the contents of the sachet are hydrophilic;
whereby, in use, the encapsulating layer is first dissolved in the gastro-intestinal tract (GIT) na d thereafter passage of the physiologically active substance into the GIT through the membrane is rate-controlled.

2. An oral delivery system according to claim 1, wherein the membrane is selected from polyethylene, polyvinyl acetate, copolymers of ethyl vinylacetate, polymethacrylate, polyvinyl chloride, ethylcelluloses, polyamides, polyurethanes, polyethers, and copolyesters.

3. An oral delivery system according to claim 1, or 2, wherein the encapsulating layer is gelatin.

4. An oral delivery system according to claim 1, wherein the sachet is enterically coated to provide a further sustained release function.

5. A method of manufacturing an oral delivery system according to claim 1, wherein two membranes are brought together at their edge regions, with a cavity being left between the edge regions, into which cavity is introduced the physiologically active substance dissolved or dispersed in the liquid or gel, the cavity sealed, and then encapsulated within a suitable encapsulating material.

6. A method according to claim 5, wherein the cavity is sealed transversely at intervals and cut in the region of the seals, so as to form individual sachets.

7-8. (canceled)
